(19) United States
(12) Patent Application Publication
(10) Pub. No.: US 2014/0146939 A1
(43) Pub. Date: May 29, 2014

(54) X-RAY DETECTOR, COLLIMATOR, CT APPARATUS AND METHOD FOR THE SAME

(71) Applicant: GE Medical Systems Global Technology Company, LLC., Waukesha, WI (US)

(72) Inventors: Xiaoyan Zhang, Beijing (CN); Xue li Wang, Beijing (CN); Jun Guo, Beijing (CN); Xuyong Yang, Beijing (CN); Qinglei Li, Beijing (CN)

(73) Assignee: GE Medical Systems Global Technology Company, LLC., Waukesha, WI (US)

(21) Appl. No.: 14/078,658

(22) Filed: Nov. 13, 2013

(30) Foreign Application Priority Data
Nov. 27, 2012 (CN) 201210489759.1

(51) Int. Cl. A61B 6/00; A61B 6/03

(52) U.S. Cl. CPC A61B 6/4208; A61B 6/035
USPC 378/19; 250/394; 378/147

(57) ABSTRACT

An X-ray detector, a collimator, a CT apparatus and a method thereof are provided. The X-ray detector comprises a plurality of detector modules which are arranged in an array along a slice direction and a signal channel direction being orthogonal to each other, the array at least comprising a left detection area, a central detection area and a right detection area that are contiguous in the signal channel direction, wherein, in the slice direction, coverage of the left detection area and coverage of the right detection area are complementary and sum of these two coverages is equal to coverage of the central detection area. The collimator comprises a pair of moving shield plates and a fixing shield plate, wherein shape of a window in the fixing shield plate is the same as shape of the array. The CT apparatus comprises the X-ray detector and the collimator.

### BACKGROUND TO THE INVENTION

The present disclosure generally relates to a CT apparatus and a method thereof, and in particular, to an X-ray detector and a collimator with special structures, a CT apparatus comprising the same and methods thereof.

### FIELD OF INVENTION

Computed tomography (CT) apparatus is more and more widely used in the medical diagnosis field as well as in other fields. Typically, CT apparatus comprises a gantry and associated controllers, an X-ray source, a collimator, an X-ray detector, a data acquisition system (DAS) and a data processing system, among others. At present, an X-ray detector and the associated DAS assemblies occupy quite a large portion of cost of a CT system. Cost is quite high because most X-ray detectors use a whole-array layout of detector modules (i.e. the signal channel is full for each layer of slices).

Furthermore, a lower X-ray dose on patients brings more benefits for patient's health during CT scanning With the technological development, to lower X-ray dose on patients has become a critical issue in the manufacture of CT apparatus.

The US Patent Application No. US2002/0071517A1 discloses a detector array. The array is symmetrical both in a slice direction and in a signal channel direction and is divided into 3 areas. The number of detector modules in the left and right areas in the slice direction is reduced as compared with that in the central area, and thus the cost is reduced. However, it also reduces the number of image slices in the left and right areas. Moreover, X-ray dose on patients is not reduced during scanning

### BRIEF DESCRIPTION OF THE INVENTION

A technical problem to be solved by the present invention is to lower cost of a CT apparatus. Another technical problem to be solved by the present invention is to lower X-ray dose on patients in CT scanning A further technical problem to be solved by the present invention is to maintain the same image quality and the number of image slices with lower cost of the CT apparatus and/or lower X-ray dose on patients. And, a still further technical problem to be solved by the present invention is to enhance the image quality of a scan object's organ of interest as compared to that of other organs.

According to a first aspect of the invention, an X-ray detector for a CT apparatus is provided, comprising a plurality of detector modules which are arranged in an array along a slice direction and a signal channel direction being orthogonal to each other, the array at least comprising a left detection area, a central detection area and a right detection area that are contiguous in the signal channel direction, wherein, in the slice direction, coverage of the left detection area and coverage of the right detection area are complementary and sum of these two coverages is equal to coverage of the central detection area.

According to one embodiment of the invention, the array further comprises a left reference area being leftmost and contiguous to the left detection area and a right reference area being rightmost and contiguous to the right detection area.

According to one embodiment of the invention, sum of number of the detector modules in the slice direction in the left detection area and number of the detector modules in the slice direction in the right detection area is M; and number of the detector modules in the slice direction in each of the central detection area, the left reference area and the right reference area is M, wherein M is an integer greater than or equal to 2.

According to one embodiment of the invention, the plurality of detector modules are identical, and wherein number of the detector modules in the slice direction in each of the left detection area and the right detection area is N, and number of the detector modules in the slice direction in each of the central detection area, the left reference area and the right reference area is 2N, N being an integer greater than or equal to 1.

According to one embodiment of the invention, the plurality of detector modules comprise long modules, left and right short modules, wherein length of a long module in the slice direction is equal to sum of lengths of the left and right short modules in the slice direction, and width of a long module in the signal channel direction is not necessarily identical to width of a left or right short module in the signal channel direction.

According to one embodiment of the invention, the length of the long module in the slice direction is a double of the length of each of the left and right short modules in the slice direction, and the width of the long module in the signal channel direction is identical to the width of each of the left and right short modules in the signal channel direction.

According to one embodiment of the invention, the left detection area and the right detection area merely comprise the short modules and number of the short modules in the slice direction is N; and the central detection area, the left reference area and the right reference area merely comprise the long modules and number of the long modules in the slice direction is also N, N being an integer greater than or equal to 1.

According to one embodiment of the invention, width of the central detection area in the signal channel direction is much smaller than width of the left detection area or the right detection area in the signal channel direction.

According to one embodiment of the invention, the width of the central detection area in the signal channel direction is reduced to zero.

According to one embodiment of the invention, there are a number of said arrays being stacked in the slice direction, and all of said arrays are aligned at both ends.

According to one embodiment of the invention, width of the central detection area in the signal channel direction depends on size of an organ of interest in a scan object.

According to one embodiment of the invention, an asymmetric algorithm is used to supplement data in an area without detector modules, based on original data obtained from a scan object by the X-ray detector or data obtained by a software algorithm.

According to one embodiment of the invention, the asymmetric algorithm and/or interpolation algorithm are/is used to supplement data in the area without detector modules, based on original data obtained from a scan object by the X-ray detector or data obtained by a software algorithm.

According to a second aspect of the invention, a collimator for a CT apparatus is provided, comprising: a pair of moving shield plates for defining a scan coverage needed for a scan object; and a fixing shield plate having a window therein, wherein shape of the window is the same as shape of the array in the X-ray detector according to any of claims 1-13, so that only the X-ray to be projected onto the array can go through the window.

According to a third aspect of the invention, a CT apparatus is provided, comprising an X-ray detector according to any of claims 1-13 and a collimator according to claim 14.

According to one embodiment of the invention, the CT apparatus further comprises an image reconstructor, wherein the image reconstructor comprises: means for using an asymmetric algorithm and/or interpolation algorithm to supplement data in an area without detector modules, based on original data obtained from a scan object by the X-ray detector or data obtained by a software algorithm; and means for reconstructing an image of the scan object based on the original data and the supplemented data.

According to a fourth aspect of the invention, a method for a CT apparatus is provided, comprising: obtaining original data from a scan object by an X-ray detector according to any of claims 1-13; supplementing data in an area without detector modules by using an asymmetric algorithm and/or interpolation algorithm; and reconstructing an image of the scan object based on the original data and the supplemented data.

The invention achieves the advantages of lowering the cost of CT apparatus, reducing X-ray dose on patients, obtaining the same or similar image quality as that of whole-array detector, and particularly enhancing the image quality of scan object's organ of interest.

### BRIEF DESCRIPTION OF DRAWINGS

The other objects, advantages and novelties of the present invention will become apparent through a detailed description of the invention with reference to the accompanying drawings, in which:

FIG. 1 is a simplified stereogram showing a CT apparatus;

FIG. 2 is a function block diagram showing the CT apparatus in FIG. 1;

FIG. 3 is a simplified structure diagram showing an X-ray detector according to a first embodiment of the invention;

FIG. 4 is a simplified structure diagram showing an X-ray detector according to a second embodiment of the invention;

FIG. 5 is a simplified structure diagram showing an X-ray detector according to a third embodiment of the invention;

FIG. 6 is a simplified structure diagram showing an X-ray detector according to a fourth embodiment of the invention;

FIG. 7 is a simplified structure diagram showing an X-ray detector according to a fifth embodiment of the invention;

FIG. 8 illustrates construction of an X-ray detector array;

FIG. 9 illustrates two image ranges provided in the field of view (FOV) according to one embodiment of the invention;

FIG. 10 illustrates a simplified stereogram of a collimator according to one embodiment of the invention;

FIGS. 11-12 illustrate a fixing shield plate of a collimator and a detector array mating with the fixing shield plate according to one embodiment of the invention;

FIG. 13 illustrates a fixing shield plate in a collimator according to another embodiment of the invention;

FIG. 14 illustrates a function of moving shield plates in a collimator according one embodiment of the invention; and

FIG. 15 illustrates a method for a CT apparatus according to one embodiment of the invention.

### DETAILED DESCRIPTION OF THE INVENTION

The present invention is described hereinafter in more detail with reference to certain embodiments and the drawings. In order to facilitate illustration rather than to be limiting, the present disclosure sets forth particulars regarding, for example, specific structures, systems, components and the like, so that persons skilled in the art can easily appreciate the present invention. However, it should be understood that the present invention can also be practiced in other embodiments without the particulars described herein, and that the solutions described herein can be completely or partially implemented by hardware and/or software (including embedded software). The invention is not limited to any specific combination of hardware and software.

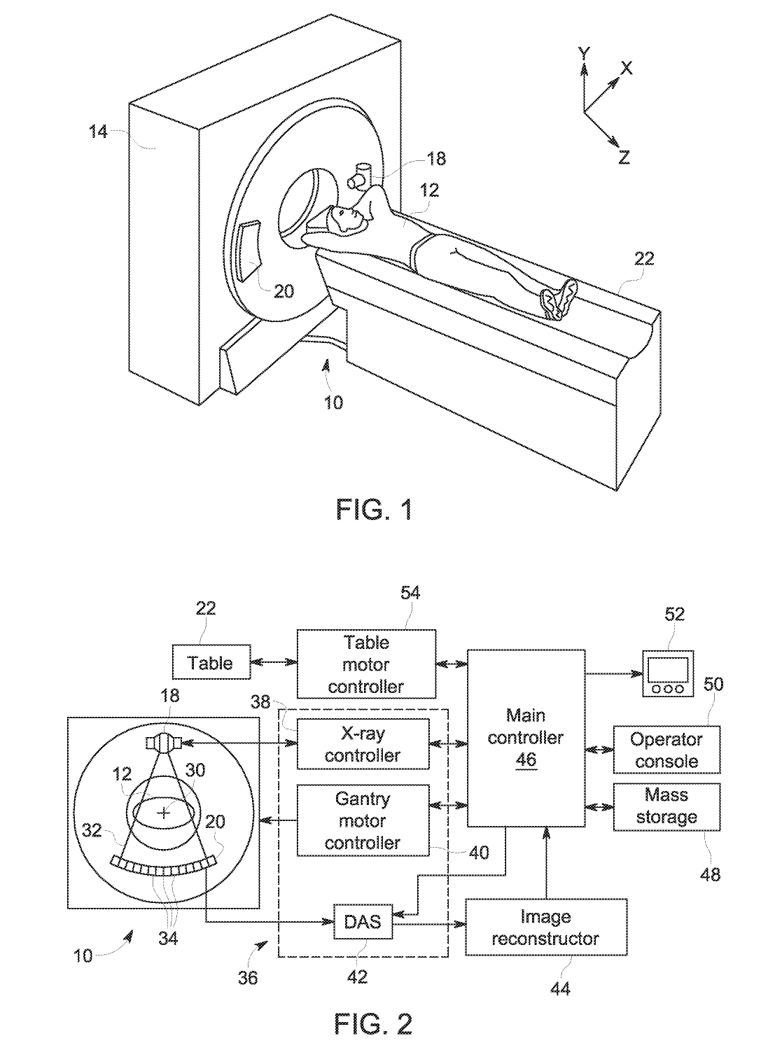

FIG. 1 is a simplified stereogram showing an exemplary CT apparatus 10 in which the invention can be embodied. The CT apparatus 10 comprises a gantry 14; an X-ray source 18, a collimator (not shown) and an X-ray detector 20 that are mounted on the gantry; a table 22 to support a scan object 12 (e.g. a patient); and other components.

As shown in FIG. 1, a direction along a body axis of the scan object 12 is typically referred to as a slice direction (or a Z-direction); an X-direction (or a signal channel direction, which is on an arc or a hypotenuse face of focal spot around an X-ray generator) is vertical to a Y-direction and defines a plane at which X-ray beams are located; both the X-direction and the Y-direction are orthogonal to the Z-direction.

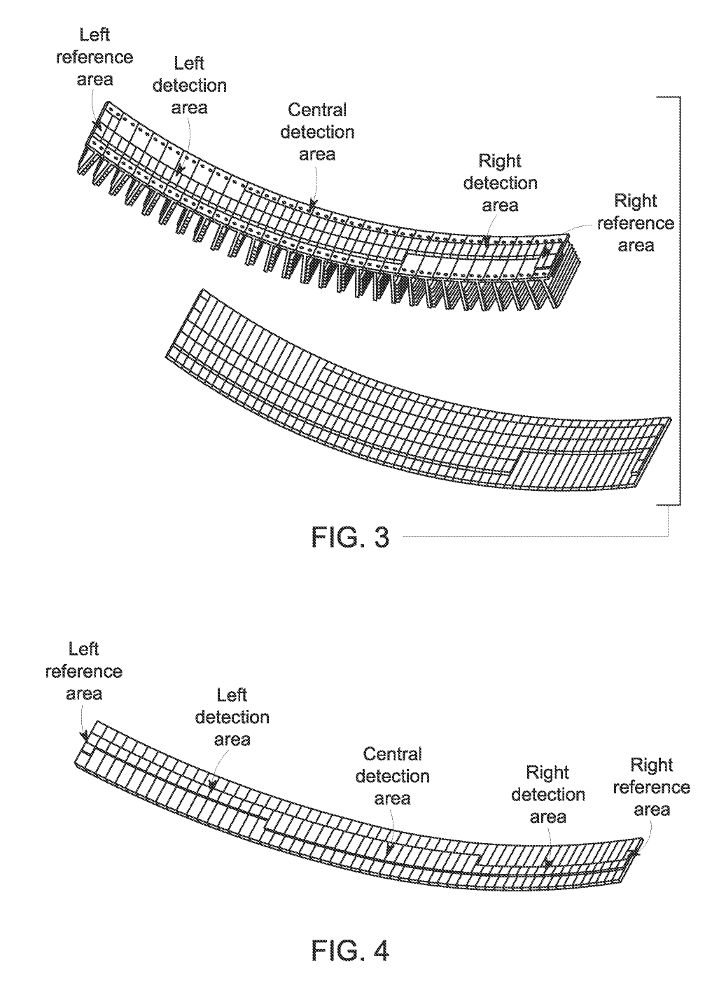

FIG. 2 is a function block diagram showing the CT apparatus 10 in FIG. 1. The table 22 translates the scan object along the Z-axis and locates an organ to be examined at a proper position under the control of a table motor controller 54. Under the control of an X-ray controller 38 and a gantry motor controller 40, an X-ray source 18 projects an X-ray beam 32 through the collimator (not shown); the X-ray beam 32 decays through the examined organ of the scan object and then projects onto the X-ray detector 20. The X-ray detector 20 comprises a plurality of detector modules 34 arranged in an array (each small block shown in FIGS. 3-5 and 7 represents one detector module). Each detector module 34 comprises a plurality of detecting elements (e.g. sensors) arranged typically in a two-dimensional subarray (each small square in FIG. 8 represents one detecting element). Each detecting element receives projected X-ray and generates electrical signal accordingly. The data acquisition system DAS 42 receives the electrical signal, generates original data by processing such as amplifying, analog-digital conversion and the like of the electrical signal, wherein data obtained from the software algorithm can be used as part of the original data, and sends the original data to an image reconstructor 44 to reconstruct image of the scan object. According to one embodiment of the invention, the image reconstructor may comprise means for using an asymmetric algorithm and/or interpolation algorithm to supplement data in an area without detector modules, based on original data obtained from a scan object by the X-ray detector, and means for reconstructing an image of the scan object based on the original data and the supplemented data. The CT apparatus 10 may further comprise a main controller 46 to control aforesaid various controllers; an operator console 50, a display 52 and a storage 48 connected with the main controller 46; and the like.

According to the invention, detector modules in an X-ray detector are arranged in a special array. In particular, a plurality of detector modules are arranged in an array along the slice direction (Z-direction) and the signal channel direction being orthogonal to each other. The array at least comprises a left detection area, a central detection area and a right detection area that are contiguous in the signal channel direction, wherein, in the slice direction (Z-direction), coverage of the left detection area and coverage of the right detection area are complementary and sum of these two coverages is equal to coverage of the central detection area. In a practical implementation, in general, for the purposes of calibration and boundary determination and the like, the array may further comprise a left reference area which is leftmost and contiguous to the left detection area and a right reference area which is rightmost and contiguous to the right detection area.

FIGS. 3-7 illustrate various embodiments of the array of detector modules according to the invention.

FIG. 3 illustrates an array of detector modules according to the first embodiment of the invention, wherein all of the detector modules are the same, and wherein the number of the detector modules in the slice direction in each of the left detection area and the right detection area is N, and the number of the detector modules in the slice direction in each of the central detection area, the left reference area and the right reference area is 2N, N being an integer greater than or equal to 1. For example, as shown in FIG. 3, N is 1 in the upper array, and N is 2 in the lower array. N can be a greater integer such as 3, 4, 5, 8, 10, 16, 32, 64, 128 and so on.

According to the invention, the number of the detector modules in the slice direction in the left detection area may be different from that in the right detection area, but the sum of said numbers in the left detection area and in the right detection area is equal to the number of the detector modules in the slice direction in the central detection area. For example, the sum of the number of the detector modules in the slice direction in the left detection area and the number of the detector modules in the slice direction in the right detection area is M; and the number of the detector modules in the slice direction in each of the central detection area, the left reference area and the right reference area is M, wherein M is an integer greater than or equal to 2.

FIG. 4 illustrates an array of detector modules according to the second embodiment of the invention, in which the plurality of detector modules comprise long modules and short modules, wherein the length of each long module in the slice direction is a double of the length of each short module in the slice direction, and the long modules and the short modules have the same width in the signal channel direction.

According to the invention, other ratios between the long modules and the short modules are possible, and are not limited to that shown in FIG. 4. For example, wherein the length of a long module in the slice direction is equal to the sum of the lengths of the left and right short modules in the slice direction, and the long modules and the short modules may have the same or different width in the signal channel direction.

Furthermore, the array in FIG. 3 may be formed by combination of the long and short modules. For example, any two adjacent modules in FIG. 3 can be replaced with one long module.

As shown in FIG. 4, the left detection area and the right detection area merely comprise the short modules and number of the short modules in the slice direction is N; and the central detection area, the left reference area and the right reference area merely comprise the long modules and number of the long modules in the slice direction is also N, N being an integer greater than or equal to 1.

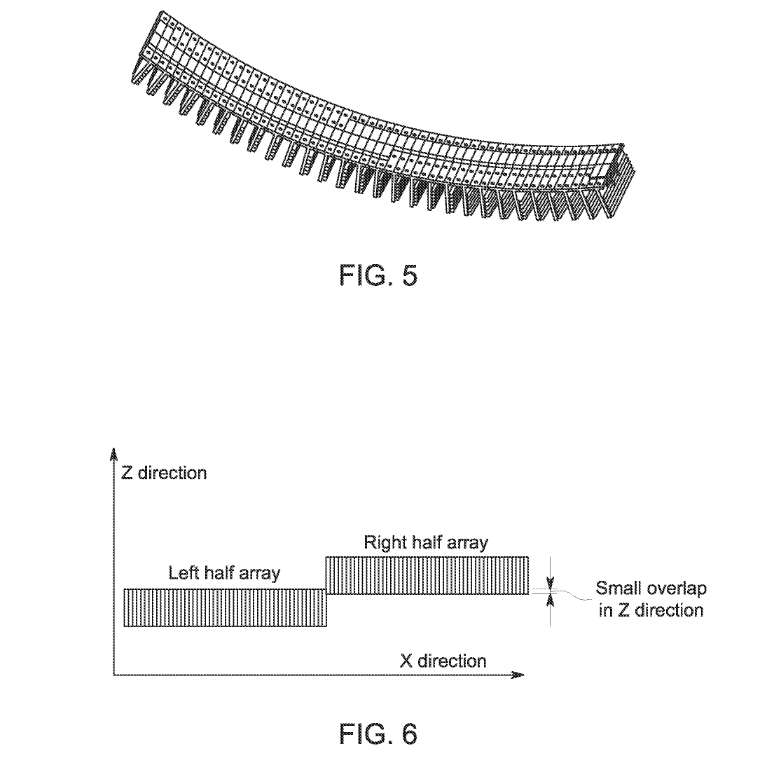

FIG. 5 illustrates an array of detector modules according to the third embodiment of the invention, wherein the width of the central detection area in the signal channel direction is much less than that of the left or right detection area. As such, the number of detector modules to be used is significantly reduced so that the manufacturing cost for such an X-ray detector is substantially reduced. It should be noted, however, that the width of the central detection area in the signal channel direction should not be less than a certain threshold value which largely depends on image reconstruction requirement.

FIG. 6 illustrates an array of detector modules according to the fourth embodiment of the invention, wherein the width of the central detection area in the signal channel direction is reduced to zero so that the array only comprises a left-half array and a right-half array. In practice, the left-half array and the right-half array generally have a small overlap along Z-direction.

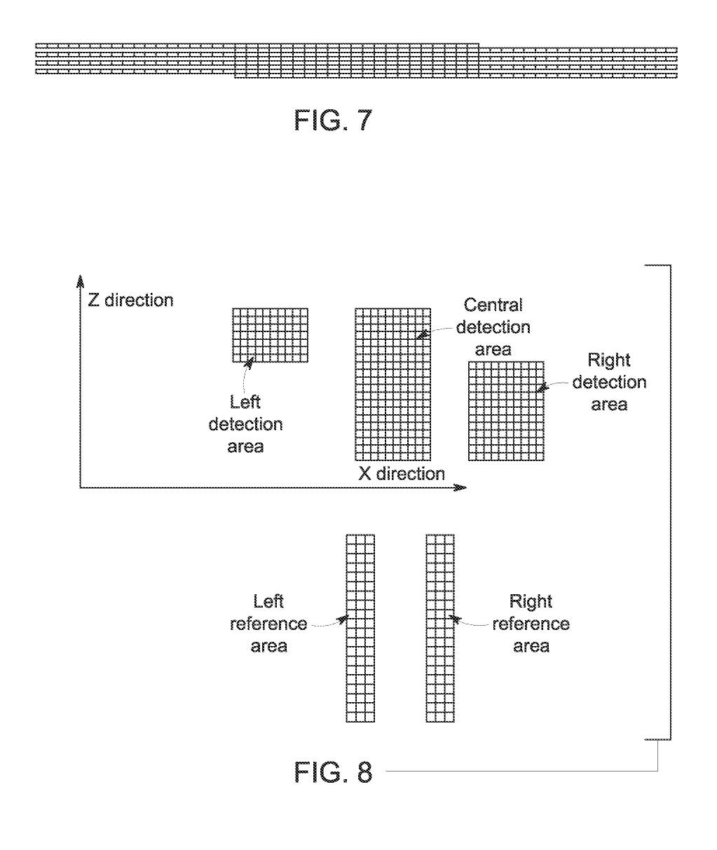

FIG. 7 illustrates an array of detector modules according to the fifth embodiment of the invention. In fact, this array is a combination of a plurality of arrays having identical structure. Each array of the first, second, third or forth embodiment can be reproduced into plurality and then the plurality of the same array are aligned at both ends and stacked along the slice direction.

FIG. 8 illustrates construction of an X-ray detector array. Each small square in FIG. 8 can represent one detecting element (the smallest detecting unit). Typically, multi-row and multi-column detecting elements are arranged in a two-dimensional subarray to form a detector module.

The number of detecting elements in Z-direction represents the number of imaging slices. In principle, the more the slices are, the higher the image quality is. According to the invention, the number of slices in the central detection area is the most, and can be designed as needed (e.g. 2, 4, 8, 10, . . . , 64, 128, 320, . . . ); FIG. 8 uses 20 slices as shown therein. The sum of the numbers (e.g. 7 and 13) of slices in the left detection area and in the right detection area is equal to the number (e.g. 20) of slices in the central detection area. The number of slices in the left/right reference area may be selected to be the same as the number of slices in the central detection area. It should be noted that the number of slices is not limited to that shown in FIG. 8, but can be 2, 3, 4, 5, 6, . . . , 16, . . . , 32, 64, 128 and the like.

According to a principle of the invention, in the slice direction (Z-direction), left detection area coverage (from a first to a seventh slice) and right detection area coverage (from a eighth to a twentieth slice) are complementary, and sum of the two coverages (a union of slices 1-7 and slices 8-20) is equal to central detection area coverage (slices 1-20).

It should be noted that the number of slices may be the same or be different in the left detection area and in the right detection area. When the numbers of slices in the left and right detection areas are the same, according to the principle mentioned above, the number of slices in each of the left and right detection areas is half of the number of slices in the central detection area.

As can be seen, an X-ray detector of the invention, when compared with a conventional whole-array X-ray detector (slices are full in each signal channel), greatly reduces the cost of a CT apparatus due to substantial reduction of the number of detector modules.

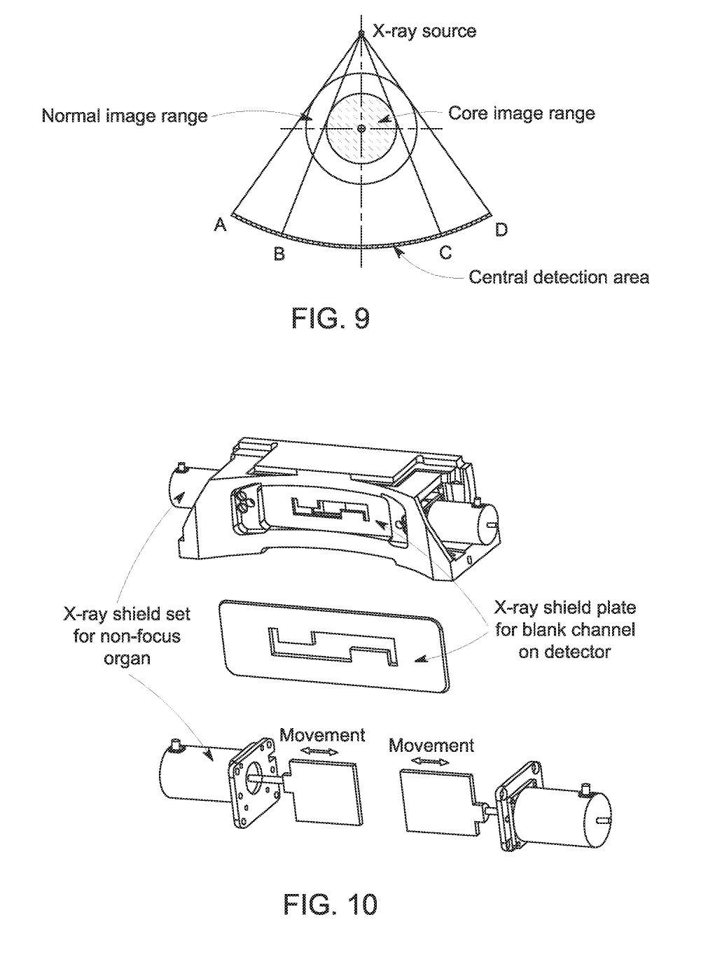

FIG. 9 shows two image ranges provided in a field of view (FOV) according to one embodiment of the invention, i.e. a core image range and a normal image range.

As shown, the core image range is located at the center of the field of view, and has a diameter D1 depending on the size of the central detection area, e.g. a width from point B to point C along the signal channel direction. Typically, the core image range should cover the scan object's organ of interest (such as heart, liver, lung and so on). In other words, in designing a detector, the width of the central detection area in the signal channel direction is mainly determined by sizes of some main organs of most scan objects.

The normal image range is an area outside the core image range and inside the field of view. The size of the normal image range depends on the sizes of the left and the right detection areas. In general, the normal image range should cover scan object's area normally scanned. In other words, in a design of the detector, the width of the left or the right detection area in the signal channel direction is mainly determined by the size of the whole body of most scan objects.

The organ of interest covered by the core image range will be imaged in a central detection area with the most slices and the better image quality; and the normally scanned area covered by the normal image range will be imaged in the left and the right detection areas with fewer slices. However, an image reconstruction method of the invention (to be elaborated later) can be used to make up data of slices being absent from the left and the right detection areas, so that the image in the left and the right detection areas can be reconstructed with almost the same image quality as that in the central detection area.

It should be noted herein that the detector array disclosed in the US Patent Application No. US2002/0071517A1 also comprises a central detection area, a left detection area and a right detection area with different quantities of slices, and scan objects' main organs are also imaged in the central detection area with the most slices. However, since the whole array is symmetrical both in a slice direction and in a signal channel direction, the special image reconstruction method of the invention cannot be applied to such a whole array to supplement missing data on the left and right, and accordingly a defect that image layers on the left and right are fewer than those in the central detection area cannot be overcome.

A conventional dissymmetrical detector needs to scan two times to achieve a scout image. However, the detector of the invention can achieve the scout image by one scan.

FIG. 10 is a simplified stereogram showing a collimator according to one embodiment of the invention. The collimator for a CT apparatus comprises: a pair of moving shield plates for defining a scan coverage needed for a scan object; and a fixing shield plate having a window therein, wherein shape of the window may be the same as shape of the array in any of the aforesaid X-ray detectors (e.g. the array in any of FIGS. 3-7), so that only the X-ray to be projected onto the array can go through the window. On the X-ray detector, X-ray will be blocked at the areas without detecting elements.

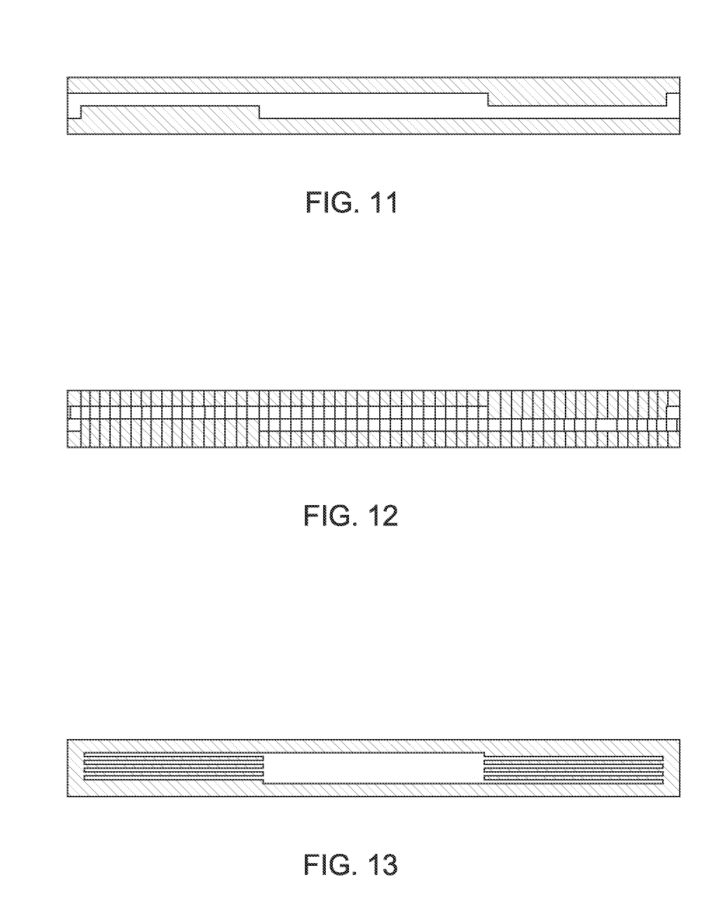

FIGS. 11-12 illustrate a fixing shield plate in a collimator and a detector array mating with the fixing shield plate according to one embodiment of the invention. The fixing shield plate may be mounted on a bottom surface of the collimator. A blank area in FIG. 11 represents a window of the fixing shield plate; the unshadowed grids in FIG. 12 represent detector modules being capable of receiving X-ray in a detector array; and the shadowed grids in FIG. 12 represent detector modules which cannot receive X-ray.

FIG. 13 illustrates a fixing shield plate in a collimator according to another embodiment of the invention, which can be cooperated with the X-ray detector array as shown in FIG. 7.

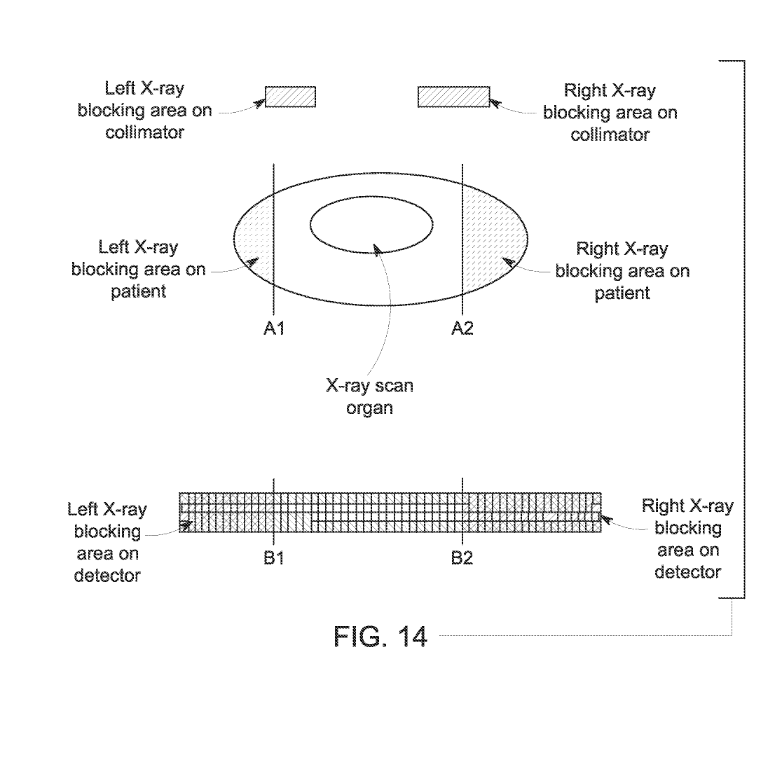

FIG. 14 illustrates functions of a moving shield plate in a collimator according to one embodiment of the invention. The moving shield plate can be driven by a motor or be regulated manually to move along the X-direction, in order to block X-ray projected towards body areas which do not need to be scanned. As indicated in FIG. 14, the areas from A1 to A2 and from B1 to B2 can be projected by X-ray. In general, the width from B1 to B2 is larger than the width from A1 to A2 because X-ray beam originated from the X-ray source is projected in a sector shape.

With use of the collimator of the invention, a moving shield plate mates with a specially-designed fixing shield plate, so that a scan object (e.g. a patient) receives far less X-ray, which is beneficial for the patient's health.

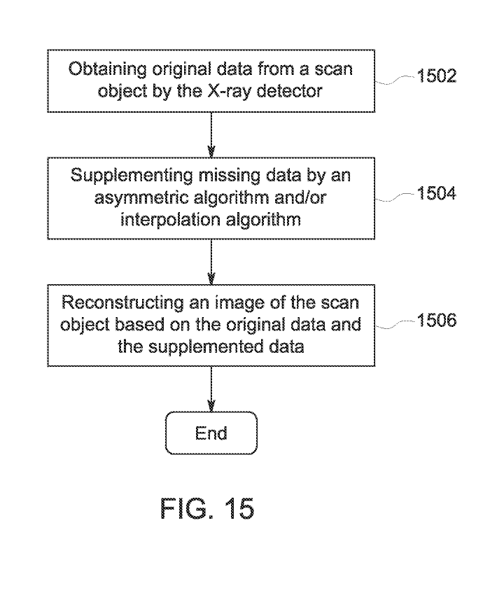

FIG. 15 illustrates a method for a CT apparatus according to one embodiment of the invention. The method comprises: obtaining original data from a scan object by the X-ray detector of the invention (step 1502); supplementing data in the area without detector modules by use of an asymmetric algorithm and/or interpolation algorithm (step 1504); reconstructing an image of the scan object based on the original data and the supplemented data (step 1506).

To be specific, the interpolation algorithm is mostly used in a plurality of stacked arrays (as shown in FIG. 7 or the overlapped arrays in FIG. 6). The Missing image data in the blank area without detector modules may be supplemented by interpolation on the basis of data obtained from two rows of modules adjacent to the blank area.

The asymmetric algorithm mainly applies for the arrays as shown in FIGS. 3-5. Each array is divided into an upper half and a lower half in the Z-direction. Each half uses the asymmetric algorithm to supplement data in the blank signal channel. For example, each array in FIG. 3 is divided into an upper half and a lower half in the Z-direction, wherein data of the left side of the upper half is supplemented by the asymmetric algorithm, and data of the right side of the lower half is supplemented by the asymmetric algorithm.

The asymmetric algorithm also applies for the stacked arrays as shown in FIG. 7, as an alternative to or in addition to the interpolation algorithm described above.

The asymmetric algorithm is detailed in Chinese Patent Application No. 201010530606.8, which is incorporated herein as a whole.

Accordingly, the detector, the collimator, the CT apparatus and the method thereof according to the invention can achieve the following technical effects:

reducing the cost of CT apparatus;

lowering X-ray dose on patients during scanning;

obtaining almost the same image quality as the whole-array detector;

enhancing especially the image quality of scan object's organ of interest.

Embodiments of the invention described herein are illustrated by way of example and not by way of limitation. Although specific terms may be adopted herein, they are only used in a general and descriptive sense, and are not for purposes of limitation. The scope of the present invention is defined only by the appended claims and equivalents thereof

This written description uses examples to disclose the invention, including the preferred embodiments, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural element with insubstantial differences from the literal languages of the claims.

What is claimed is:

1. An X-ray detector for a CT apparatus, comprising:
a plurality of detector modules which are arranged in an array along a slice direction and a signal channel direction being orthogonal to each other, the array at least comprising a left detection area; and
a central detection area and a right detection area that are contiguous in the signal channel direction, wherein, in the slice direction, coverage of the left detection area and coverage of the right detection area are complementary and sum of these two coverages is equal to coverage of the central detection area.

2. The X-ray detector according to claim 1, wherein the array further comprises a left reference area being leftmost and contiguous to the left detection area; and
a right reference area being rightmost and contiguous to the right detection area.

3. The X-ray detector according to claim 2, wherein a sum of the plurality of detector modules in the slice direction in the left detection area and number of the plurality of detector modules in the slice direction in the right detection area is M; and a number of the plurality of detector modules in the slice direction in each of the central detection area, the left reference area and the right reference area is M, wherein M is an integer greater than or equal to 2.

4. The X-ray detector according to claim 3, wherein each detector module of the plurality of detector modules is identical, and wherein the number of the detector modules in the slice direction in each of the left detection area and the right detection area is N, and number of the detector modules in the slice direction in each of the central detection area, the left reference area and the right reference area is 2N, N being an integer greater than or equal to 1.

5. The X-ray detector according to claim 2, wherein the plurality of detector modules comprise long modules, and left and right short modules, wherein length of a long module in the slice direction is equal to sum of lengths of the left and right short modules in the slice direction, and a width of a long module in the signal channel direction is not necessarily identical to a width of a left or right short module in the signal channel direction.

6. The X-ray detector according to claim 5, wherein the length of the long module in the slice direction is double of the length of each of the left and right short modules in the slice direction, and the width of the long module in the signal channel direction is identical to the width of each of the left and right short modules in the signal channel direction.

7. The X-ray detector according to claim 5, wherein the left detection area and the right detection area merely comprise the short modules and number of the short modules in the slice direction is N; and
the central detection area, the left reference area and the right reference area merely comprise the long modules and number of the long modules in the slice direction is also N, N being an integer greater than or equal to 1.

8. The X-ray detector according to claim 1, wherein a width of the central detection area in the signal channel direction is much smaller than a width of the left detection area or the right detection area in the signal channel direction.

9. The X-ray detector according to claim 8, wherein the width of the central detection area in the signal channel direction is reduced to zero.

10. The X-ray detector according to claim 4, wherein there are a number of said arrays being stacked in the slice direction, and all of said arrays are aligned at both ends.

11. The X-ray detector according to claim 1, wherein a width of the central detection area in the signal channel direction depends on size of an organ of interest in a scan object.

12. The X-ray detector according to claim 1, wherein an asymmetric algorithm is used to supplement data in an area without the plurality of detector modules, based on original data obtained from a scan object by the X-ray detector or data obtained by a software algorithm.

13. The X-ray detector according to claim 10, wherein the asymmetric algorithm and/or interpolation algorithm are/is used to supplement data in the area without the plurality of detector modules, based on original data obtained from a scan object by the X-ray detector or data obtained by a software algorithm.

14. A collimator for a CT apparatus, comprising:
a pair of moving shield plates for defining a scan coverage needed for a scan object; and
a fixing shield plate having a window therein, wherein shape of the window is the same as shape of the array in the X-ray detector according to claim 1—so that only the X-ray to be projected onto the array can go through the window.

15. A CT apparatus, comprising an X-ray detector according to claim 1 and a collimator according to claim 14.

16. The CT apparatus according to claim 15, further comprising an image reconstructor, wherein the image reconstructor comprises:
means for using an asymmetric algorithm and/or interpolation algorithm to supplement data in an area without detector modules, based on original data obtained from a scan object by the X-ray detector or data obtained by a software algorithm; and means for reconstructing an image of the scan object based on the original data and the supplemented data.

17. A method for a CT apparatus, comprising:
obtaining original data from a scan object by the X-ray detector according to claim 1;
supplementing data in an area without detector modules by using an asymmetric algorithm and/or interpolation algorithm; and reconstructing an image of the scan object based on the original data and the supplemented data.
